(19) United States
(12) Patent Application Publication
(10) Pub. No.: US 2003/0165581 A1
(43) Pub. Date: Sep. 4, 2003

(54) Compositions for treating diabetes mellitus, methods of use and manufacturing process of the same

(76) Inventors: Peng Wang, Burlingame, CA (US); Lin Lei, Melshan (CN)

Correspondence Address: WILSON SONSINI GOODRICH & ROSATI, 650 PAGE MILL ROAD, PALO ALTO, CA, 943041050

(21) Appl. No.: 10/091,371

(22) Filed: Mar. 4, 2002

(51) Int. Cl. A61K 35/78

(52) U.S. Cl. 424/725

(57) ABSTRACT

The present invention provides novel compositions and methods for lowering blood glucose levels, as well as manufacture processes for producing the compositions. Specifically, the present invention provides novel compositions that are extracts of the plant Prunella Linn and/or Rabdosis (Blume) Hasskarl containing enriched corosolic acid. Methods of isolating corosolic acid at high purity from these plants are also provided. These extracts and the purified corosolic acid can be used for lowering blood sugar levels and reducing accumulation of triglyeride in the treatment of diabetes, obesity and related conditions.

### BACKGROUND OF THE INVENTION

1. Field of the Invention

This invention relates to composition and methods for lowering blood glucose, and, more particularly, to extracts from Prunella Linn or Rabdosis (Blume) Hasskarl that contains corosolic acid and to methods of purifying corosolic acid from these extracts.

2. Description of Related Art

Diabetes mellitus is an insidious disease for which there is presently no cure. Mammals afflicted with diabetes mellitus will, unless the glucose level in the blood is controlled, ultimately suffer heart attacks, strokes, loss of eyesight, loss of limbs and ultimately may die as the result of this disease. Diabetes affects more than million people in the United States and is the fourth leading cause of death. Diabetes is also the principle cause of blindness in adults and is the most common cause of kidney failure. Neuropathy, artery disease and premature aging are common conditions associated with chronically elevated blood sugar level.

Generally, there are two major forms of diabetes mellitus: insulin-dependent (type-I) and noninsulin-dependent diabetes mellitus (type-II). Type I diabetes, also called juvenile-onset diabetes mellitus, most often strikes suddenly in childhood. Type-I diabetes affects only about 5% of the diabetic population. In contrast, type-II diabetes, also called maturity-onset diabetes mellitus, usually develops rather gradually after the age of 40. In recent years, there has been an increase in the incidence of type-II diabetes, especially in developed countries such as the United States.

The polypeptide hormone insulin acts mainly on muscle, liver, and adipose tissue cells to stimulate the synthesis of glycogen, fats, and proteins while inhibiting the breakdown of these metabolic fuels. In addition, insulin stimulates the uptake of glucose by most cells, with the notable exception of brain and liver cells. Together with glucagon, which has largely opposite effects, insulin acts to maintain the proper level of blood glucose.

In diabetes, insulin either is not secreted in sufficient amounts or does not efficiently stimulate its target cells. As a consequence, blood glucose levels become so elevated that the glucose “spills over” into the urine, providing and convenient diagnostic test for the disease. Yet, despite of these high blood glucose levels, cells “starve” since insulin-stimulated glucose entry into the cells is impaired. Triacylglycerol hydrolysis, fatty acid oxidation, glucogeogenesis, and ketone body formation are accelerated, which eventually causes a decrease in blood volume, and ultimately life-threatening situations.

In type-I diabetes, insulin is absent or nearly so because the pancreas lacks or has defective β cells. This condition results from an autoimmune response that selectively destroy the β cells. Individuals with insulin-dependent diabetes requires regular insulin injections to survive and must follow carefully balanced diet and exercise regimens.

Insulin-dependent diabetics must have insulin administered to them in a very rigorous, disciplined manner and must have snacks between meals since it is necessary to maintain the proper level of insulin in the bloodstream, i.e., undesirable side effects are experienced if the insulin level is too high and the disease will continue unabated if the insulin level is too low. In addition, a disciplined diet is required and if the patient is unwilling or not able to accept insulin injections, pharmaceutical preparations such as “Diabinase”, “Orinase”, “Glynase”, “Glucophage”, etc. must be taken. All in all, the insulin-dependent patient is constantly on the narrow edge of either too much or insufficient medication and frequently is not able to tolerate such medication because of its side effects.

Type-II diabetes or non-insulin-dependent diabetes mellitus, accounts for over 90% of the diagnosed cases of diabetes and affects more than 16 million people in the US and some 200 million people around the world. Yousef et al. (1999) Diabetes Review 7: 55-76. Contrasting with type I diabetes, type II diabetic individuals have normal or even greatly elevated insulin levels. Their symptoms arise from an apparent paucity of insulin receptors on normally insulin-responsive cells. It has been hypothesized that the increased insulin production resulting from overeating, consequently obesity, eventually, suppresses the synthesis of insulin receptor.

Type II diabetes causes various disabling microvascular complications in patients. Besides retinopathy, nephropathy, and neuropathy, the disease is also associated with accelerated atherosclerosis and premature cardiovascular morbidity and mortality. This increased incidence of atherosclerosis (e.g., myocardial infarction, stroke, and peripheral vascular disease) is intricately associated with insulin resistance, which is a major pathophysiologic abnormality in type II diabetes. The insulin resistance of type II diabetes contributes to the metabolic abnormalities of hyperglycemia, hyperinsulinemia, dyslipidemia, hypertension, and hypercoaglulation.

Type-II diabetes sufferer must follow a disciplined program of diet and exercise to avoid the necessity of taking medication to control blood glucose levels. However, many non-insulin dependent diabetes sufferers experience difficulty in conscientiously following such program and will ultimately fall into the insulin-dependent category sooner or later.

There are numerous side effects, discomfort and inconvenience associated with long-term injections of insulin. Overdosing or mismanagement of the administration may lower the blood glucose level to such a dangerous level that results in hypoglycemic shock or tic. Thus, there is a long-felt need for hypoglycemic agents that are natural, holistic edible, and capable of restoring the blood glucose level of a diabetes sufferer to the normal levels. In addition, there is also a need for manufacturing processes for producing such hypoglycemic agents efficiently and cost-effectively.

### SUMMARY OF THE INVENTION

The present invention provides novel compositions and methods for lowering blood glucose levels as well as manufacture processes for producing the compositions.

In one aspect of the present invention, compositions are provided for lowering blood glucose levels. The composition comprises: extract of a plant Prunella Linn and/or Rabdosis (Blume) Hasskarl containing corosolic acid (or 2α-hydroxyursolic acid) at a concentration of at least 0.01%, preferably at least 0.1%, more preferably at least 1% and most preferably at least 10% by weight.

The whole plant or the portion grown above the ground of Prunella Linn and/or Rabdosis (Blume) Hasskarl may be extracted.

The extract may further comprise ursolic acid, 2α,19α-dihydricursolic acid or daucosterol.

The composition may be formulated in a pharmaceutically acceptable carrier for oral administration to a human subject. Such carriers includes tablets, pills, dragees, capsules, emulsions, lipophilic and hydrophilic suspensions, liquids, gels, syrups, slurries, suspensions and the like, for oral ingestion by a patient to be treated.

In a preferred embodiment, the composition is contained in capsules. Capsules suitable for oral administration include push-fit capsules made of gelatin, as well as soft, sealed capsules made of gelatin and a plasticizer, such as glycerol or sorbitol. The push-fit capsules can contain the extract in admixture with a filler such as lactose, binders such as starches, and/or lubricants such as talc or magnesium stearate and, optionally, stabilizers.

More preferably, the inventive composition is contained in soft capsules and dissolved or suspended in suitable liquids, such as fatty oils or liquid polyethylene glycols. The fatty oil may be any natural or synthetic oil suitable for oral administration to a human. Examples of natural oil include, but are not limited to corn oil, wheat germ oil, soy bean oil, rice bran oil, rapeseed oil, sesame oil, fish oil and other vegetable and animal oils. In addition, stabilizers may be added. All formulations for oral administration should be in dosages suitable for such administration.

In another aspect of the present invention, methods are provided for reducing the blood glucose of a mammal, preferably a human. The method comprises: administering to the mammal a hypoglycemically effective amount of an extract of a plant Prunella Linn and/or Rabdosis (Blume) Hasskarl containing corosolic acid at a concentration of at least 0.01%, preferably at least 0.1%, more preferably at least 1% and most preferably at least 10% by weight.

The method may be used to lower blood glucose levels of a human suffering from type I or type II diabetes and/or obesity. The method may also be used to lower blood glucose levels of a human in situations of acute stress such as experienced by animals or patients with hyperthermia, trauma, sepsis, and burns and undergoing general anesthesia. The method may also be used to treat hyperglycemia associated with severe head injury, cerebral thrombosis, encephalitis and heat stroke.

The extract may be administered alone or combined with any physiologically acceptable carrier such as water, an aqueous solution, normal saline, or other physiologically acceptable excipient.

The amount of corosolic acid in the extract administered to a human subject is preferably about 10-500 mg per day, more preferably about 20-100 mg per day, and most preferably 30-50 mg per day.

The extracts of the present invention can be administered by a number of routes, including, but not limited to: orally, injection including, but not limited to intravenously, intraperitoneally, subcutaneously, intramuscularly, etc. The preferred route of administration is oral.

Optionally, the extract may be administered in conjunction with another hypoglycemic including such as insulin; a biguanide such as metformin or buformin; a sulfonylurea such as acetohexamide, chlorpropamide, tolazamide, tolbutamide, glyburide, glypizide or glyclazide; a thiazolidinedione such as troglitazone; an α-glucosidase inhibitor such as acarbose or miglatol; or β₃-adrenoceptor agonist such as CL-316, 243, etc.

The extract may be administered after being converted to pharmaceutically acceptable salts using a counter ion such as sodium, potassium, lithium, calcium, magnesium, zinc or iron.

In yet another aspect of the present invention, a method is provided for manufacturing extract of a plant Prunella Linn and/or Rabdosis (Blume) Hasskarl containing corosolic acid.

The method comprises: extracting a plant material from Prunella Linn and/or Rabdosis (Blume) Hasskarl in a first solvent such that the resulting extract contains corosolic acid at a concentration of at least 0.01%, preferably at least 0.1%, more preferably at least 1% and most preferably at least 10%.

The method may further comprise: grinding the whole plant material or the portion grown above the ground.

The first solvent may be polar solvent. Suitable polar solvents include, but are not limited to, methanol, ethanol, 2-methoxyethanol, 1-propanol, 2-propanol, iso-butanol, sec-butanol, tetrahydrofuran, other polar solvents know to those skilled in the art, and mixtures thereof.

Preferably, the first solvent used to extract Prunella Linn or Rabdosis (Blume) Hasskarl is methanol or ethanol, and more preferably >90% ethanol.

The ratio of the plant material and the solvent is preferably between 1:3 to 1:20, more preferably between 1:5 to 1:10, and most preferably between 1:6 to 1:20.

The plant material may be extracted for about 1-24 hours, more preferably preferably for about 3-10 hours, and most preferably for about 4-6 hours at room temperature, or heated at a temperature from about room temperature to about the reflux temperature for the first solvent.

The method may optionally further comprise: decolorizing the extract to reduce the amount of chlorophyll in the extract, for example, by using activated carbon.

The method may optionally further comprise: partitioning the extract between the first solvent and an aliphatic solvent such as petroleum ether, No. 120 solvent gasoline or a mixture of both to reduce the amount of aliphatic molecules in the extract.

The method may optionally further comprise: partitioning the extract in a biphasic mixture of a second polar and second non-polar solvent to yield a crude extract of corosolic acid at concentration of at least 0.1%.

Examples of the second polar solvent include, but are not limited to, methanol, ethanol, acetone, 1-propanol, 2-propanol, iso-butanol, sec-butanol, tetrahydrofuran, or a mixture thereof.

Example of the second non-polar solvent include, but are not limited to, diethyl ether, ethyl acetate, isoamyl acetate, benzene, toluene, xylene, 2-butanone, 4-methyl-2-pentanone, chlorinated hydrocarbons such as dichloromethane, chloroform, carbon tetrachloride, 1,2-dichloroethane, tetrachloroethylene, petroleum ether, and a mixture thereof.

In a particular embodiment, the second polar solvent is ethanol and the second non-polar solvent is chloroform, and the chloroform phase containing the extracted corosolic acid is retained. In another embodiment, the second polar solvent is acetone and the second non-polar solvent is chloroform, and the acetone phase containing the extracted corosolic acid is retained.

The method may further comprise: purifying corosolic acid from the extract. According to the method, corosolic acid may be purified by chromatography such as thin-layer chromatography, conventional silica gel chromatography, vacuum flash chromatography, high performance liquid chromatography, and combinations thereof. Each of the purification methods may be performed more than once. In a particular embodiment, the chromatography is silica gel chromatography. The eluent solvent for the silica gel chromatography includes, but is not limited to, chloroform:acetone at a ratio of 60˜90:40˜10.

The method may further comprise: crystallizing corosolic acid in the extract such that the purity of corosolic acid is at least 50%, preferably at least 80%, more preferably at least 90%, and most preferably at least 98%.

### BRIEF DESCRIPTION OF THE FIGURES

FIG. 1 shows the chemical structures of corosolic acid, ursolic acid, 2α,19α-dihydricursolic acid.

FIG. 2 shows the chemical structure of daucosterol.

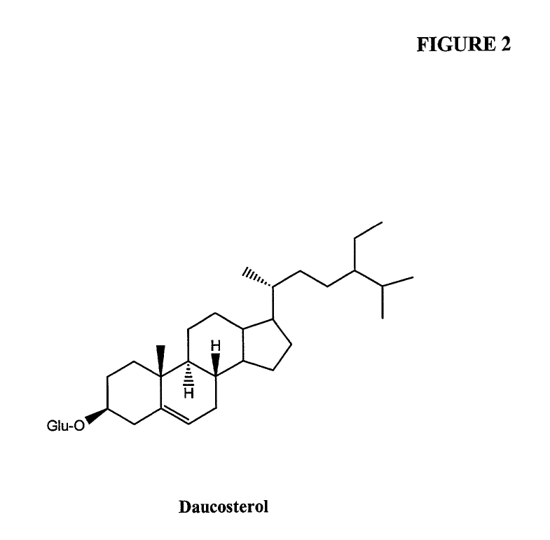

FIG. 3 is a flow chart of an embodiment of the process for extracting and purifying corosolic acid from Prunella Linn or Rabdosis (Blume) Hasskarl.

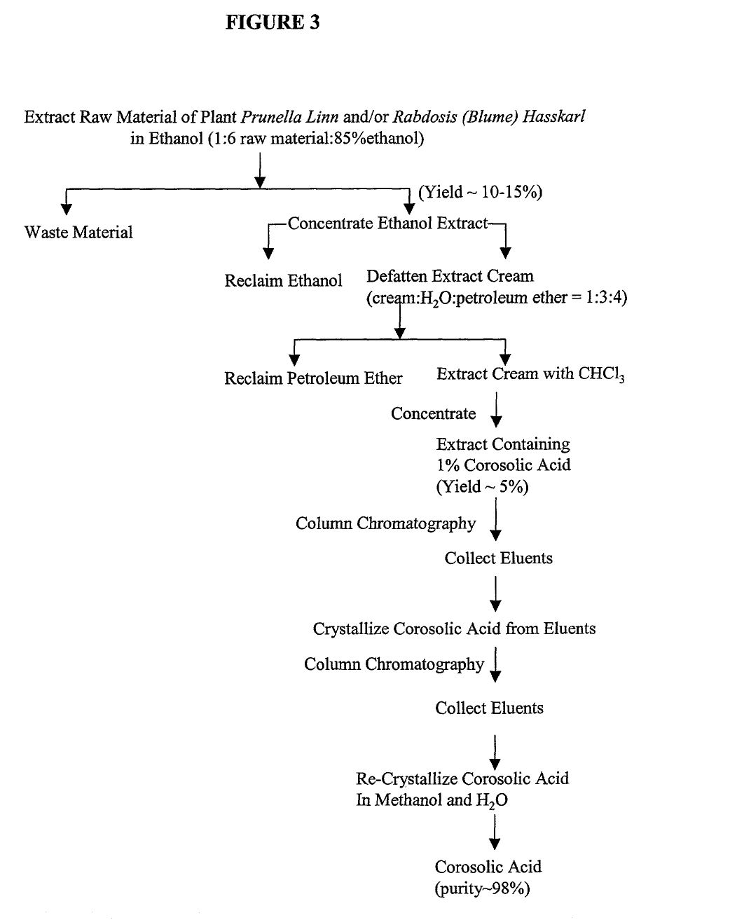

### DETAILED DESCRIPTION OF THE INVENTION

The present invention provides novel compositions and methods for lowering blood glucose levels as well as manufacture processes for producing the compositions. Specifically, the present invention provides novel compositions that are extracts of the plant Prunella Linn and/or Rabdosis (Blume) Hasskarl containing enriched corosolic acid. Methods of isolating corosolic acid at high purity from these plants are also provided. These extracts and the purified corosolic acid can be used for lowering blood sugar levels and reducing accumulation of triglyeride in the treatment of diabetes, obesity and related conditions.

1. Corosolic Acid and Its Use in Diabetes Treatment

1) Corosolic Acid

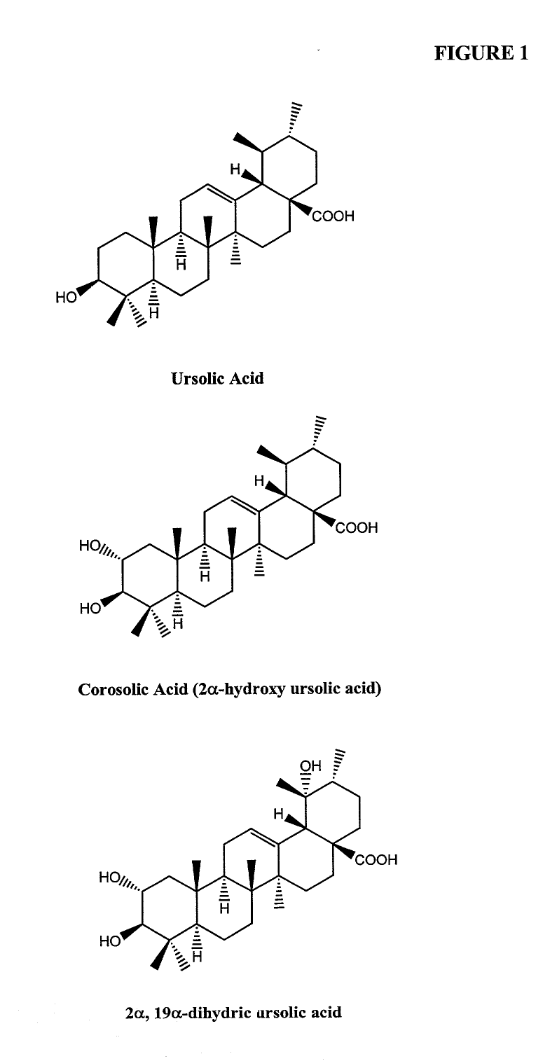

Corosolic acid, or 2α-hydroxyursolic acid, is a triterpenoid compound with its chemical structure shown in FIG. 1. Corosolic acid has been found to be able to activate the transport of glucose across cell membranes, resulting blood sugar reduction. With such an activity similar to that of insulin, a hormone that naturally increases glucose transport activity across the cell membrane, corosolic acid is also coined the “phyto-insulin”.

Corosolic acid possesses many advantages over insulin in safety, pharmacokinetics and routes of administration. Oral administration of insulin does not reduce blood sugar, whereas orally administered corosolic acid can produce a drop in blood sugar levels. Large doses of injected insulin are capable of producing adverse reactions, while oral doses of corosolic acid have no known side effects. In rabbits, oral doses of corosolic acid have been shown to act similarly to subcutaneous injections of insulin.

2) Preclinical Studies

Preclinical studies have demonstrated that corosolic acid is able to activate cell glucose-transporter shuttles and thus helps balance blood glucose levels. Glucose transport is the most important way that cells acquire energy. An increase of glucose transport through the cell membrane facilitates the lowering of blood sugar. Therefore, finding a safe activator of glucose transport is crucial to the Type II diabetic. Ehrlich ascites tumor cells are useful for screening the glucose transport potential of natural products. In one study, the time course of glucose uptake by Ehrlich cells was measured and corosolic acid showed significant glucose transport-stimulating activity. Murakami et al. (1993) Chem. Pharm. Bull. (Tokyo) 41:2129-2131.

The sugar-lowering effects of corosolic acid were then studied in hereditary Type II diabetic mice. Kaduda et al. Biosci. Biotechnol. Biochem. (1996) 60:20-208. In the first experiment, one group of mice was fed a control diet while the other group was given a diet that included corosolic acid for a period of five weeks. The plasma glucose levels increased in the control group, but this increase was completely suppressed in the mice given corosolic acid. In this experiment, crossover of the diet between the two groups yielded results consistent with the above observation. In a second experiment on Type II diabetic rats, supplementation with corosolic acid resulted in a decrease in glucose levels whereas the control group showed an elevation of blood sugar levels. The level of serum insulin, urinary excreted glucose and total plasma cholesterol were also lowered in mice supplemented with corosolic acid.

In another study using normal rabbits, a baseline analysis of initial blood sugar in 24 hour-fasted rabbits was conducted. Following administration of corosolic acid, blood glucose was analyzed at one, two, three, four and five hours. The results indicated that a large oral dose of corosolic acid produced blood sugar reductions similar to the effects of two units of insulin. Large doses of corosolic acid caused a more than 57 mg blood sugar reduction per 100 mL blood, i.e., a reduction of 57 mg/dL in blood sugar concentration. Oral administration of corosolic acid reduced blood sugar in normal rabbits in amounts ranging from 16 to 49 mg per 100 mL blood. A repetition of the first dose after two more hours caused the blood sugar to remain low (or go even lower than the first dose) for more than 5 hours. Larger doses of corosolic acid caused a 40 to 58 mg blood glucose reduction per 100 mL blood. The peak reduction, after large doses, occurred from two to four hours after the administration of corosolic acid, and the blood sugar returned to normal in 6 to 10 hours. Thus, significant and immediate blood sugar reduction was observed in response to varying doses of corosolic acid.

In 1991, researchers at an Italian university observed that an oral dose of corosolic acid reduces blood sugar levels in mice. Similarly, Dr. K. Osawa at Tohoku University, Japan reported that corosolic acid reduced blood sugar levels from 300 mg/dL to 150 mg/dL in rats with experimentally induced diabetes. This study showed that corosolic acid induces a blood glucose lowering effect as immediate as an injection of insulin.

3. Clinical Studies

Clinical studies of corosolic acid on human subjects were conducted in Japan and the United States. In 1998, a crossover, placebo-controlled clinical study was conducted at the Tokyo Jikeikai Medical School in Japan with 24 human subjects. The criteria for including the subjects in this study were a mild case of Type II diabetes, inability to tolerate a high glucose burden, glucose levels of 100 mg per dL (fasting level) and subjects older than 20 years of age. The subjects were given orally either a placebo or a standardized corosolic acid tablet after each meal three times daily. The results of this study clearly demonstrate that corosolic acid is effective in reducing blood glucose levels in short-term (four weeks) treatment, with no signs of adverse effects. Furthermore, even a one-time dose leaves a “memory-effect” for blood glucose control, for a few days. Compared to the placebo group, a statistically significant drop in the average blood glucose level is observed with the administration of corosolic acid.

In 1999, a clinical study was conducted by Dr. William V. Judy at the Southwestern Institute of Biomedical Research, Brandenton, Fla., to confirm corosolic acids' effect in lowering blood glucose levels and to evaluate the dose-response relationship. The randomized, double-blind, cross over trial was conducted with 12 subjects (6 women and 6 men) over 22 weeks. The criteria for including subjects in this study were mild Type II diabetes, inability to tolerate a high glucose burden, glucose levels of more than 150 mg/dL (fasting level) and subjects older than 46 years of age with an informed consent. The clinical reference value of normal blood glucose ranges from 65 to 110 mg/dL.

Corosolic acid, in an oil based soft gelatin capsule, was given to each group of people at the dose of 16, 32 or 48 mg per day for two weeks. The average blood glucose level dropped 4.9% at 16 mg, while the decrease was 10.7% at 32 mg, and a drop of 31.9% was noted at 48 mg per day of corosolic acid.

The second group of five people was given corosolic acid, formulated in a dry powder base, in two-piece hard gelatin capsule, at 16, 32 or 48 mg corosolic acid per day. In this group, compared to the placebo, the average blood glucose level dropped by 3.18% at 16 mg, 6.5% at 32 mg, and 20.2% at a 48 mg daily dose of corosolic acid.

These results indicate that the higher the daily dose of corosolic acid, the greater the drop in blood glucose levels. Furthermore, an oil-based soft gelatin capsule formulation of corosolic acid seems to be more potent than a comparable dry-powder formulation over the same dose range. These results suggest differences in absorption with significantly greater blood glucose reduction at a 48 mg daily dose of corosolic acid, in an oil-based soft gelatin formulation.

The subjects were monitored for various parameters: blood glucose, blood pressure, body weight, temperature, heart rate and general health and comfort in response to the supplement. Patient feed back was also noted.

In the cross-over study, a group of 12 subjects was given a placebo for two weeks and their fasting blood glucose levels was monitored. The same group was given an oral daily dose of 48 mg corosolic acid (two capsules of 8 mg corosolic acid after each meal or a total of six capsules a day), in an oil-based soft gelatin formulation, for a period of 30 days. A (placebo) washout period of 45 days followed. After the washout period, the same group was crossed over to a daily 48 mg corosolic acid treatment (two capsules of 8 mg corosolic acid after each meal or a total of 6 capsules a day), in dry powder hard gelatin formulation, for a period of 30 days.

After the hard gelatin corosolic acid treatment, a second washout period of 45 days followed. The blood glucose levels were monitored at 15-day intervals, during the dosing and washout periods.

The results of this cross-over study demonstrate that an oral dose of corosolic acid is effective in reducing blood glucose levels, with no signs of adverse effects. The average blood glucose level in the control group was 168.3 mg/deciliter. The soft gelatin formulation of corosolic acid caused a rapid drop to an average value of 127.2 and 115.1 mg/deciliter at the 15th and 30th day of corosolic acid treatment, respectively. During the washout period, the recovery of the blood glucose level was slow (131.7, 153.2 and 168.2 mg/dL at 15, 30 and 45 days of the washout period). The washout period blood glucose levels suggest a memory effect of corosolic acid for up to four weeks, after the termination of the treatment.

These results indicate that 48 mg of corosolic acid per day shows a continued blood glucose reduction until the end of the 30-day period. Corosolic acid supplementation seems to help in regaining blood glucose control in adult onset diabetes (Type-II) compared to no treatment in the control phase. Steeper decline in blood glucose levels and maintenance of lower blood glucose levels are evident in corosolic acid supplementation compared to control conditions.

Furthermore, corosolic acid treatment causes a sharper decline in blood glucose levels after a meal, resembling a normoglycemic profile, compared to the slow decline after a meal observed in (diabetic) untreated control conditions. Subjects under corosolic acid supplementation report the disappearance of conditions associated with adult onset diabetes, such as frequent thirst and urination.

Subjects receiving the oil-based corosolic acid formulation in a soft gelatin capsule seem to show an increased tendency toward weight loss (an average weight loss of 3.2 pounds), compared to those on the dry-powder based corosolic acid formulation (no weight loss).

Corosolic acid is clinically proven to activate cell glucose-transporter “shuttles” and thus helps balance blood glucose levels. Corosolic acid shows a memory effect of blood glucose lowering even after the treatment is stopped. An oil-based corosolic acid formulation in a soft gelatin capsule seems to be relatively more efficient in lowering blood glucose levels, perhaps through increased absorption from the gut into the bloodstream.

These latest U.S. clinical study results confirm the 1998 Japanese clinical study showing that corosolic acid safely and effectively lowers blood glucose levels in Type II diabetics.

Corosolic acid also delivers a strong antioxidant activity to scavenge free radicals and to prevent cell membrane lipid peroxidation. In addition, corosolic acid helps maintain low blood pressure and normal kidney function, by controlling blood sugar, and thus preventing damage to blood vessels and kidneys.

2. Novel Plant Extract Containing Enriched Corosolic Acid

Corosolic acid used in preclinical and clinical studies described above is extracted or purified from leaves of Lagerstroemia speciosa L., a tree from southern Asia. However, extraction and purification of corosolic acid from this plant may not meet the demand for large quantity of this drug for large scale clinical trials and world wide commercialization. Excessive harvesting of the leaves of the tree can pose a threat to the environment and ecological balances of plant species.

The present invention provides a novel method for extracting and purifying corosolic acid from Prunella Linn or Rabdosis (Blume) Hasskarl. Unlike Lagerstroemia speciosa L. which is a tree growing slowly, both Prunella Linn and Rabdosis (Blume) Hasskarl are perennial herb with a short growth cycle and can be harvested year round. For Lagerstroemia speciosa L. only the leaves can be extracted to produce corosolic acid; whereas for Prunella Linn and Rabdosis (Blume) Hasskarl the whole plant or the part above the ground can be used for producing a large quantity of extracts containing enriched corosolic acid or purified corosolic acid.

The family of Prunella Linn includes 7-15 species that can be found in the temperate and tropical zones of Europe and Asia, Northwestern Africa and North America. The family of Rabdosis (Blume) Hasskarl includes 2 species: X and Y. The X species of Rabdosis (Blume) Hasskarl can be found Eastern China; and the Y species in Southern and Southwestern China.

3. Process for Preparing Hypoglycemically Active Extracts and/or Corosolic Acid

According to the present invention, the hypoglycemic compound corosolic acid employed in the methods and pharmaceutical compositions of the present invention can be isolated from Prunella Linn or Rabdosis (Blume) Hasskarl, either as components of hypoglycemically active extracts, or in substantially purified form, using the illustrative methods described below.

The hypoglycemically active extract contains corosolic acid, preferably at least 0.1% by weight, more preferably at least 1% by weight, and most preferably at least 10% by weight.

The substantially purified form of corosolic acid is purified from the crude materials or crude extracts of Prunela Linn or Rabdosis (Blume) Hasskarl containing corosolic acid of at least 1% by weight. In The substantially purified form of corosolic acid, purity of corosolic acid is preferably at least 50% by weight, more preferably at least 85% by weight, and most preferably at least 98% by weight.

Prior to extraction, whole plant material from Prunella Linn or Rabdosis (Blume) Hasskarl is optionally be ground to powder or otherwise reduced in overall size, so as to increase the effective surface area of the plant material available to the solvent during extraction.

The plant material from Prunella Linn or Rabdosis (Blume) Hasskarl is extracted with a first solvent to obtain a solution of one or more hypoglycemically active compounds including corosolic acid. Depending on the species, the plant material is taken from the whole plant or above the ground part.

The first solvent may be a polar solvent. Suitable polar solvents include, but are not limited to, methanol, ethanol, 2-methoxyethanol, 1-propanol, 2-propanol, iso-butanol, sec-butanol, tetrahydrofuran, other polar solvents know to those skilled in the art, and mixtures thereof. The polar solvent may optionally be diluted with water in order to adjust the polarity thereof. In this case, the aqueous content of the polar solvent can range from 0 to about 50%, preferably from 0 to about 20%.

Preferably, the first solvent used to extract corosolic acid from Prunella Linn or Rabdosis (Blume) Hasskarl is a polar solvent, more preferably, methanol or ethanol, and most preferably, >90% ethanol.

The ratio of the plant material and the solvent is preferably between 1:3 to 1:20, more preferably between 1:5 to 1:10, and most preferably between 1:6 to 1:20.

The extraction of plant material can be facilitated by placing it in a suitable vessel with the first solvent, and allowing the mixture to stir preferably for about 1-24 hours, more preferably preferably for about 3-10 hours, and most preferably for about 4-6 hours. The first solvent can be at room temperature, or heated at a temperature from about room temperature to about the reflux temperature for the particular solvent system employed.

The solid residue is filtered and may be extracted again under the conditions described above to yield more extract solution. The resulting extract solutions containing corosolic acid are combined and concentrated, optionally in vacuum, to provide an enriched mixture containing corosolic acid and one or more other hypoglycemically active compounds, such as ursolic acid, 2α,19α-dihydricursolic acid, and daucosterol. The extract solution can optionally be filtered through, e.g., conventional filter paper, celite, or a small layer of silica gel, prior to concentration. The resulting enriched extract may optionally be subjected to decoloration, such as by activated carbon, to rid of chlorophyll. The resulting enriched extract may also be subjected to partitioning between the first solvent and an aliphatic solvent such as petroleum ether, No. 120 solvent gasoline or a mixture of both at a 1˜2:1 ratio, to rid of aliphatic molecules in the extract.

The enriched extract is subjected to the step of partitioning the enriched extract in a biphasic mixture of a second polar and second non-polar solvent to rid of aliphatic molecules, yielding a crude extract of corosolic acid.

Suitable second polar solvents include, but are not limited to, methanol, ethanol, acetone, 1-propanol, 2-propanol, iso-butanol, sec-butanol, tetrahydrofuran, other polar solvents known to those skilled in the art, and mixtures thereof. Suitable second non-polar solvents include, but are not limited to, diethyl ether, ethyl acetate, isoamyl acetate, benzene, toluene, xylene, 2-butanone, 4-methyl-2-pentanone, chlorinated hydrocarbons such as dichloromethane, chloroform, carbon tetrachloride, 1,2-dichloroethane, tetrachloroethylene, petroleum ether, and other non-polar solvents known to those skilled in the art, and mixtures thereof.

Although the partitioning step can be performed more than once, the same solvent system, i.e., the biphasic mixture of the second polar solvent and second non-polar solvent, needn't be used in each iteration. In other words, different polar solvents can be used in conjunction with different non-polar solvents in each iteration of the partitioning step.

In a particular embodiment, the biphasic mixture of the first iteration comprises ethanol as the second polar solvent and chloroform as the second non-polar solvent at about 2:1 weight ratio, and the biphasic mixture of the second iteration comprises about acetone as the second polar solvent and chloroform as the second non-polar solvent at about 2:1 weight ratio.

It is to be understood that the first and second polar solvents, and first and second non-polar solvents are independent of each other, such that the first polar solvent need not be the same as the second polar solvent, and the first non-polar solvent need not be the same as the second non-polar solvent.

After the partitioning, the extract containing corosolic acid in the second polar solvent may be further concentrated to yield an extract concentrate.

Optionally, the extract concentrate can be further purified to obtain corosolic acid in substantially purified form. Suitable methods of purification include, but are not limited to, recrystallization from solvents and solvent mixtures known to those skilled in the art, elution chromatography and combinations thereof. Methods of elution chromatography include, but are not limited to, preparative thin-layer chromatography, conventional silica gel chromatography, vacuum flash chromatography, high performance liquid chromatography, and combinations thereof. Each of the purification methods can be performed more than once, if necessary.

In a preferred embodiment, the extract concentrate obtained as described above is purified using using conventional silica gel chromatography to provide corosolic acid in substantially purified form. The eluent from the chromatography that contains corosolic acid in substantially purified form may also include other structurally similar compounds, such as ursolic acid, 2α,19α-dihydricursolic acid and daucosterol. This eluent may be evaporated to crystallize the mixture containing corosolic acid in substantially purified form.

Depending on the purity of corosolic acid desired, the chromatographic purification step may be repeated at least once to further separate corosolic acid from other structurally similar compounds and the eluent is subjected to recrystallization to yield highly pure corosolic acid crystal, preferably with purity higher than 98%.

4. Methods for Using Corosolic Acid in Extract or in Substantially Purified Form

Both corosolic acid in substantially purified form and in extracts of Prunella Linn or Rabdosis (Blume) Hasskarl containing corosolic acid prepared using the methods described above have hypoglycemic activity. Due to the potent activity of the corosolic acid-containing extracts of the present invention, the extracts are advantageously useful in veterinary and human medicine for therapeutic treatment of diabetes mellitus.

Additionally, the extracts can be advantageously be used as hypoglycemic agents to reduce the blood glucose level in situations of acute stress such as experienced by animals or patients with hyperthermia, trauma, sepsis, and burns and undergoing general anesthesia. Hyperglycemia sometimes associated with severe head injury, cerebral thrombosis, encephalitis and heat stroke can also be therapeutically treated with these extracts. Additionally, the extracts are useful as hypoglycemic agents for rare congenital metabolic glycogen storage disease associated with hyperglycemia.

Although not wishing to be limited by any particular mechanism of action to explain the hypoglycemic activity of the corosolic acid-containing extracts of the present invention, it is envisaged that they may advantageously be useful for treatment of both insulin-dependent or type I diabetes (formerly termed juvenile-onset or ketosis-prone diabetes) and non-insulin-dependent or type II diabetes (formerly termed adult-onset, maturity-onset or nonketotic diabetes).

When administered to a mammal for veterinary use or to a human for clinical use, the extracts of the present invention can be used alone, or may be combined with any physiologically acceptable carrier such as water, an aqueous solution, normal saline, or other physiologically acceptable excipient. In general, the amount of corosolic acid in the extract administered to the subject is preferably about 10-500 mg per day, more preferably about 20-100 mg per day, and most preferably 30-50 mg per day.

The extracts of the present invention can be administered by a number of routes, including, but not limited to: orally, injection including, but not limited to intravenously, intraperitoneally, subcutaneously, intramuscularly, etc. The preferred route of administration is oral.

For oral administration, the extract of the present invention can be formulated readily by combining with pharmaceutically acceptable carriers that are well known in the art. Such carriers enable the compounds to be formulated as tablets, pills, dragees, capsules, emulsions, lipophilic and hydrophilic suspensions, liquids, gels, syrups, slurries, suspensions and the like, for oral ingestion by a patient to be treated.

In a preferred embodiment, the extract of the present invention is contained in capsules. Capsules suitable for oral administration include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. More preferably, the inventive composition is contained in soft capsules. The extract of the present invention, if in solid form, may be dissolved or suspended in suitable liquids, such as fatty oils or liquid polyethylene glycols. The fatty oil may be any natural or synthetic oil suitable for oral administration to a human. Examples of natural oil include, but are not limited to corn oil, wheat germ oil, soy bean oil, rice bran oil, rapeseed oil, sesame oil, fish oil and other vegetable and animal oils. In addition, stabilizers may be added. All formulations for oral administration should be in dosages suitable for such administration.

Optionally, the extract of the present invention for oral use can be obtained by mixing the inventive compositioon with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

For buccal administration, the extract of the present invention may take the form of tablets or lozenges formulated in conventional manner.

For administration by inhalation, the extract of the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas, or from propellant-free, dry-powder inhalers. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

Additionally, the extracts of the present invention can be administered in conjunction with another hypoglycemic including such as insulin; a biguanide such as metformin or buformin; a sulfonylurea such as acetohexamide, chlorpropamide, tolazamide, tolbutamide, glyburide, glypizide or glyclazide; a thiazolidinedione such as troglitazone; an α-glucosidase inhibitor such as acarbose or miglatol; or β₃-adrenoceptor agonist such as CL-316, 243, etc.

The extracts of the present invention can be administered in an effective amount either as isolated form as described above or can be converted to pharmaceutically acceptable salts using a counter ion such as sodium, potassium, lithium, calcium, magnesium, zinc or iron.

In addition, the extracts of Prunella Linn or Rabdosis (Blume) Hasskarl containing corosolic acid or pharmaceutically acceptable salts thereof can be used for research purposes, for example, to investigate the mechanism and activity of hypoglycemic agents.

The compositions provided by the present invention can be advantageously useful in veterinary and human medicine for therapeutic treatment of diabetes mellitus. Additionally, extracts of and purified corosolic acid from Prunella Linn or Rabdosis (Blume) Hasskarl can be advantageously used as hypoglycemic agents to reduce the blood glucose level in situations of acute stress such as experienced by animals or patients with hyperthermia, trauma, sepsis, and burns and undergoing general anesthesia. Hyperglycemia sometimes associated with severe head injury, cerebral thrombosis, encephalitis and heat stroke can also be therapeutically treated with these compositions. Additionally, these compositions or compounds are useful as hypoglycemic agents for rare congenital metabolic glycogen storage disease associated with hyperglycemia.

Although not wishing to be limited to any particular mechanism of action to explain the hypoglycemic activity of extracts containing corosolic acid or corosolic acid in substantially purified form, the inventors envisage that they may advantageously be useful for treatment of both insulin-dependent or type I diabetes and non-insulin-dependent or type II diabetes.

The composition of the present invention may be conveniently used by hyperglycemic people to reduce blood sugar levels without causing many side effects and inconvenience of administration associated with the use of insulin. It may be significant that extracts from natural herb Prunella Linn or Rabdosis (Blume) Hasskarl that includes not only corosolic acid but also other compounds such as ursolic acid, 2α,19α-dihydricursolic acid and daucosterol may have synergistic effects on hyperglycemic and/or obese people by targeting different path ways of glucose transportation and fat storage and metabolism. This feature is particularly advantageous for treating heavy-set persons, those who suffer side effects when taking insulin or synthetic hypoglycemic preparations, and those whose life styles are such that they are unwilling or unable to adhere to a rigorous exercise/diet program throughout their lives.

### EXAMPLES

The following is an example of manufacturing process of herbal extracts containing enriched corosolic acid and further purification of corosolic acid from the extracts.

### 1. Extraction of Prunella Linn and Rabdosis (Blume) Hasskarl to Yield Crude Extracts Containing Enriched Corosolic Acid

Whole plant of Prunella Linn or Rabdosis (Blume) Hasskarl was harvested, washed and dried until water consent was reduced below 10%. The dried plant was ground to powder.

To a 2000 L extraction vessel was added 300 Kg of dried plant power and 1800 Kg of ethanol (>90% in concentration). The mixture was stirred under reflux temperature of ethanol for 5 hr. The extract solution was collected. The solid residue was extracted the second time with 1800 Kg of >90% ethanol for 4 h and the third time for 2 hr. The extract solutions collected from these three extractions were combined and concentrated, yielding an extract cream.

The extract cream was mixed with water and petroleum ether (60- 90° C.) at a ratio of 1:3:4 to rid of aliphatic molecules at 20-60° C. After the solution separated from petroleum ether was then partitioned in chloroform at 20-30° C. for 2-4 hr for three times. The chloroform solution was concentrated to yield a crude extract cream containing about 1% corosolic acid. The whole process can be carried out efficiently and on a large scale without going through column chromatography, thus particularly desirable for industrial production. The yield of this crude extract cream isolated from the raw plant materials was about 5%.

### 2. Purification of Corosolic Acid from Crude Extract of Prunella Linn and Rabdosis (Blume) Hasskarl

The crude extract containing enriched corosolic acid at about 1% was further purified using vacuum flash chromatography. The crude extract was dissolved in methanol and filtered to rid of undissolved residue. The extract/methanol solution was mixed with G100˜200 silica gel at a weight ratio of 1:1.5 and then dried until the weight was substantially constant. This mixture of G100˜200 silica was loaded on top of G200˜300 silica gel at a weight ratio of 1:1.5 in a column. The loaded column was non-gradient-washed with chloroform:acetone (60˜90:40˜10). Fractions of eluent were analyzed by TLC to identify the portions containing corosolic acid. The fractions of eluent containing high concentration of corosolic acid were pooled, concentrated, and then dissolved in methanol at a weight ratio of 1:1.5˜2.

The fractions of eluent containing lower concentration of corosolic acid were collected and subjected to the second chromatography by using G200˜300 silica gel column and gradient-washing with chloroform:isopropanol (99˜97:1˜3). Similar to the first chromatography, the fractions of eluent containing high concentration of corosolic acid were pooled and combined with the pooled fractions of eluent from the first chromatography. The fractions of eluent containing less concentrated corosolic acid were subjected to the third chromatography by following the same procedure as the second chromatography.

The pooled fractions of eluent containing high concentration of corosolic acid was filtered to rid of undissolved residue and subjected to crystallization in methanol:water. 30˜40% methanol water solution was gradually added into the pooled eluents until orange crystals precipitated out. A slight amount of methanol was added into the mixture which was stored at 0˜10° C. to allow further crystallization of the corosolic acid. This process yielded crude crystals containing ≧60% corosolic acid mixed with structurally similar compounds such as ursolic acid, 2α,19α-dihydricursolic acid and daucosterol.

The crude crystals containing ≧60% corosolic acid were further purified to yield corosolic acid with ≧98.25% purity. The fractions of eluent containing corosolic acid were collected, concentrated and dissolved in acetone. The acetone solution was subjected to recrystallization in 30˜40% methanol water solution, yielding white corosolic acid crystals with ≧98.25% purity. The yield of corosolic acid purified from the 1% crude extract cream was about 75%.

The ≧98.25% corosolic acid crystals produced in the above-described process were characterized by using standard methods and compared with a corosolic acid standard (C₃₀H₄₈O₄). The melting point was determined to be 242-244° C. and did not decrease when the sample was mixed with the corosolic acid standard. Mass spectroscopy (set at a high resolution power electron impact scanning mode, 70 ev) revealed the following peaks: 472 (M⁺), 454 (M⁺−H₂O), 442 (M⁺−2×CH₃), (M⁺−2×H₂O), 426, 408, 393, 370, 300, 287, 264, and 248. The NMR spectrum contained the following peaks: δH 5.52 (1H, W/Z=7 Hz, 12-H), 4.15 (1H, m, 2β-H), 3.42 (1H, d, J=9 Hz, 3α-H), 1.28 (6H, S, 2×CH₃), 1.22, 1.09, 1.06, 1.02, 0.99 (each 3H, S, 5×CH₃). The IR spectrum contained the following peaks: 3430-3390, 1690, 1450, 1383, 1372, 1045, 1028, 955 γ_{MAX}cm⁻¹. All of the characteristics of the corosolic acid purified by using the inventive method are consistent with those of the corosolic acid standard.

What is claimed is:

1. A composition comprising: extract of a plant Prunella Linn or Rabdosis (Blume) Hasskarl containing corosolic acid at a concentration of at least 0.01% by weight.

2. The composition of claim 1, wherein the concentration of corosolic acid is at least 0.1% by weight.

3. The composition of claim 1, wherein the concentration of corosolic acid is at least 1% by weight.

4. The composition of claim 1, wherein the concentration of corosolic acid is at least 10% by weight.

5. The composition of claim 1, wherein the extract is an extract of the whole plant of Prunella Linn or Rabdosis (Blume) Hasskarl.

6. The composition of claim 1, wherein the extract is an extract of the portion of the plant that grows above the ground.

7. The composition of claim 1, further comprising:
ursolic acid, 2α,19α-dihydricursolic acid or daucosterol.

8. The composition of claim 1, wherein the corosolic acid is in a form of solid.

9. The composition of claim 1, wherein the extract is in a form of liquid.

10. A pharmaceutically acceptable composition, comprising:
a pharmaceutically acceptable excipient; and
extract of a plant Prunella Linn or Rabdosis (Blume) Hasskarl containing corosolic acid at a concentration of at least 0.01% by weight.

11. The composition of claim 10, wherein the pharmaceutically acceptable composition is suitable for oral administration to a human.

12. The composition of claim 10, wherein the pharmaceutically acceptable composition is formulated with the excipient in a form selected from the group consisting of tablets, pills, dragees, capsules, emulsions, lipophilic and hydrophilic suspensions, liquids, gels, syrups, slurries, and suspensions.

13. The composition of claim 12, wherein the pharmaceutically acceptable composition is formulated in hard or soft-gel capsules.

14. The composition of claim 10, wherein the excipient is selected from the group consisting of glycerol, sorbitol, lactose, magnesium stearate, maize starch, wheat starch, rice starch, potato starch, gelatin, gum tragacanth, methyl cellulose, hydroxypropylmethyl-cellulose, sodium carboxymethylcellulose, and polyvinylpyrrolidone.

15. The composition of claim 10, wherein the excipient is an pharmaceutically acceptable oil.

16. The composition of claim 15, wherein the pharmaceutically acceptable oil is selected from the group consisting of corn oil, wheat germ oil, soy bean oil, rice bran oil, rapeseed oil, sesame oil, and fish oil.

17. The composition of claim 10, wherein the concentration of corosolic acid is at least 1% by weight.

18. The composition of claim 10, wherein the concentration of corosolic acid is at least 10% by weight.

19. The composition of claim 10, further comprising:
ursolic acid, 2α,19α-dihydricursolic acid or daucosterol.

20. A method for lowering blood sugar levels of a mammal, comprising:
administering to the mammal a hypoglycemically effective amount of an extract of a plant Prunella Linn or Rabdosis (Blume) Hasskarl containing corosolic acid at a concentration of at least 0.01% by weight.

21. The method of claim 20, wherein the concentration of corosolic acid is at least 1% by weight.

22. The method of claim 20, wherein the concentration of corosolic acid is at least 10% by weight.

23. The method of claim 20, wherein the concentration of corosolic acid is at least 50% by weight.

24. The method of claim 20, wherein the extract is orally administered to the mammal.

25. The method of 20, wherein the extract administered to the mammal via inhalation.

26. The method of claim 20, wherein the mammal is a human.

27. The method of claim 26, wherein the human has a condition selected from the group consisting of hyperglycermia, hyperinsulinemia, dyslipidemia, hypertension, hypercoaglulation, obesity, type I and type II diabetic mellitus.

28. The method of claim 26, wherein the extract is administered to the human to deliver corosolid acid in an amount of 10-500 mg per day.

29. The method of claim 26, wherein the extract is administered to the human to deliver corosolid acid in an amount of 20-100 mg per day.

30. The method of claim 26, wherein the extract is administered to the human to deliver corosolid acid in an amount of 30-50 mg per day.

31. The method of claim 20, further comprising:
administering to the mammal another hypoglycemic agent selected from the group consisting of insulin, metformin, buformin, sulfonylurea, acetohexamide, chlorpropamide, tolazamide, tolbutamide, glyburide, glypizide, glyclazide, thiazolidinedione, troglitazone, acarbose, miglatol, CL-316 and CL-243.

32. The method of claim 20, wherein the extract is administered after being converted to pharmaceutically acceptable salts using a counter ion.

33. The method of claim 32, wherein the counter ion is selected from the group consisting of sodium, potassium, lithium, calcium, magnesium, zinc and iron.

34. The method for manufacturing an extract of a plant Prunella Linn or Rabdosis (Blume) Hasskarl containing corosolic acid, comprising:
extracting a plant material from Prunella Linn or Rabdosis (Blume) Hasskarl in a first polar solvent such that the resulting extract contains corosolic acid at a concentration of at least 0.01%.

35. The method of claim 34, wherein the concentration of corosolic acid is at least 1% by weight.

36. The method of claim 34, wherein the concentration of corosolic acid is at least 10% by weight.

37. The method of claim 34, wherein the concentration of corosolic acid is at least 50% by weight.

38. The method of claim 34, wherein the first solvent is an aqueous solution or organic solvent.

39. The method of claim 34, wherein the first polar solvent is selected from the group consisting of methanol, ethanol, 2-methoxyethanol, 1-propanol, 2-propanol, iso-butanol, sec-butanol, tetrahydrofuran, and a mixture thereof.

40. The method of claim 34, wherein the first polar solvent is a mixture of ethanol and water at a weight ratio of 3:1 to 10:1.

41. The method of claim 34, wherein the first polar solvent is ethanol with purity of at least 95%.

42. The method of claim 34, further comprising: grind the whole plant material or the portion grown above the ground.

43. The method of claim 34, wherein the ratio of the plant material and the solvent is between 1:3 to 1:20 by weight.

44. The method of claim 34, wherein the ratio of the plant material and the solvent is between 1:5 to 1:10 by weight.

45. The method of claim 34, wherein the ratio of the plant material and the solvent is between 1:6 to 1:20 by weight.

46. The method of claim 34, wherein the plant material is extracted by heating for about 1-24 hours at the reflux temperature of the first solvent.

47. The method of claim 34, wherein the plant material is extracted by heating for about 3-8 hours at the reflux temperature of the first solvent.

48. The method of claim 34, further comprising: decolorizing the extract to reduce the amount of chlorophyll in the extract.

49. The method of claim 48, wherein the extract is decolorized by using activated carbon.

50. The method of claim 34, further comprising:
partitioning the extract between the first solvent and an aliphatic solvent to reduce the amount of aliphatic molecules in the extract.

51. The method of claim 50, wherein the aliphatic solvent is petroleum ether, solvent gasoline or a mixture thereof.

52. The method of claim 34, further comprising:
partitioning the extract in a biphasic mixture of a second polar and second non-polar solvent to yield a crude extract of corosolic acid at concentration of at least 0.1%.

53. The method of claim 52, wherein the second polar solvent is selected from the group consisting of methanol, ethanol, acetone, 1-propanol, 2-propanol, iso-butanol, sec-butanol, tetrahydrofuran, and a mixture thereof.

54. The method of claim 52, wherein the second non-polar solvent is selected from the group consisting of diethyl ether, ethyl acetate, isoamyl acetate, benzene, toluene, xylene, 2-butanone, 4-methyl-2-pentanone, chlorinated hydrocarbons such as dichloromethane, chloroform, carbon tetrachloride, 1,2-dichloroethane, tetrachloroethylene, petroleum ether, and a mixture thereof.

55. The method of claim 52, wherein the second polar solvent is ethanol and the second non-polar solvent is chloroform, and the chloroform phase containing the extracted corosolic acid is retained.

56. The method of claim 52, wherein the second polar solvent is acetone and the second non-polar solvent is chloroform, and the acetone phase containing the extracted corosolic acid is retained.

57. The method of claim 34, further comprising:
purifying corosolic acid from the extract.

58. The method of claim 57, wherein corosolid acid is purified by chromatography.

59. The method of claim 58, wherein the chromatography is selected from the group consisting of thin-layer chromatography, conventional silica gel chromatography, vacuum flash chromatography, high performance liquid chromatography, and combinations thereof.

60. The method of claim 58, wherein the chromatography is silica gel chromatography and the eluent solvent for the chromatography is chloroform:acetone at a ratio of 60˜90:40˜10.

61. The method of claim 34, further comprising:
crystallizing corosolic acid in the extract such that the purity of corosolic acid is at least 50%.

62. The method of claim 34, further comprising: crystallizing corosolic acid in the extract such that the purity of corosolic acid is at least 80%.

63. The method of claim 34, further comprising: crystallizing corosolic acid in the extract such that the purity of corosolic acid is at least 90%.

64. The method of claim 34, further comprising: crystallizing corosolic acid in the extract such that the purity of corosolic acid is at least 98%.
